(19) United States
(12) Patent Application Publication
(10) Pub. No.: US 2024/0043784 A1
(43) Pub. Date: Feb. 8, 2024

(54) BIOLOGICAL SPECIMEN INCUBATOR

(71) Applicant: Analog Devices, Inc., Wilmington, MA (US)

(72) Inventors: Max Marcel Marcel Shulaker, Cambridge, MA (US); Alicia Ann Coto, Boston, MA (US); Graham E. McGrath, Boston, MA (US); Samuel H. Fuller, Wellesley, MA (US)

(21) Appl. No.: 18/229,636

(22) Filed: Aug. 2, 2023

Related U.S. Application Data
(60) Provisional application No. 63/370,226, filed on Aug. 2, 2022.

(51) Int. Cl. C12M 1/00; C12M 1/34; C12M 1/36; C12M 1/02

(52) U.S. Cl. CPC C12M 41/14; C12M 41/34; C12M 41/48; C12M 27/00

(57) ABSTRACT

An incubation system for gas detection of a biological specimen can include a gas or optical sensor. The sensor can be arranged to be placed in communication with at least one biological specimen vessel for generating an electrical response signal indicating a chemical characteristic associated with the specimen. The specimen can be incubated in a temperature-controlled chamber, such as within a shelf defining a plurality of receptacles. Processing circuitry can be included or used such as to place the sensor and at least one biological specimen vessel in communication with each other.

### CLAIM FOR PRIORITY

This application claims priority to U.S. Provisional Application Ser. No. 63/370,226, filed Aug. 2, 2022, which is hereby incorporated by reference herein in its entirety.

### BACKGROUND

Blood incubation can be used to help detect foreign bodies, such as bacteria or other microorganisms, within the bloodstream of a human patient. For example, a blood sample can be taken from the patient and incubated to determine whether an infection exists. During an incubation period, certain microorganism cultures can multiply within the blood, enabling several techniques to detect infections. For example, a noticeable change in the sample's properties can develop throughout incubation. The sample can be analyzed to help form a medical diagnosis of the patient.

Incubation periods of a blood sample can range between about four hours to multiple days, depending upon the type of microorganism and the ambient temperature. The sample can be maintained at approximately body temperature (about 37° C.), or the temperature of the sample can be established such as to enhance growth of a certain target foreign body. Such incubation processes can involve combining the sample with a fluid, such as an nutrient liquid or surface that is enriched for microorganism growth within a test tube or Petri dish. A blood sample can be separated into several components (e.g., plasma, red blood cells, white blood cells, platelets, etc.) during collection. Any of these components can be used in similar techniques of blood incubation. Also, other body fluids can be used for similar incubation and analysis, such as spinal fluid, synovial fluid, cerebrospinal fluid, sweat, urine, and saliva.

### SUMMARY

Certain biological specimen incubation techniques involve several manual operations to perform during incubation, or in between incubation sessions. Manual intervention of incubation of a specimen can present challenges, such as difficulty in obtaining repeatable results and possible operator-introduced errors. Further, an incubation technique can involve visual inspection of biological specimens for signs of an infection, such as observing for signs of a change in color, clarity, size, or other characteristics of the specimen. Such visual inspection can introduce contamination or lead to operator-introduced errors in determining whether a target foreign body is present within the biological specimen. The present inventors have recognized a need for a more user-friendly, reproducible, less operator-dependent, more sterile, and more cost-efficient technique for biological specimen incubation. This document describes an incubation system for gas detection of at least one incubated biological specimen, the system including at least one gas sensor, arranged to be placed in communication with at least one biological specimen vessel for generating an electrical response signal indicating a chemical characteristic associated with the specimen.

A biological specimen incubator can include or use a temperature-controlled chamber to perform incubation of biological specimen therein. The chamber can include or use at least one shelf defining at least one receptacle, and the at least one receptacle can be sized and shaped such as for receiving at least one biological specimen vessel. In an example, the incubator can include or use processing circuitry to place at least one sensor and an individual vessel of the at least one biological specimen vessels in communication with each other. For example, the processing circuitry can help determine, using at least one electrical response signal from the at least one sensor, a presence or other characteristic of at least one target gas composition associated with the particular biological specimen. In an example, the at least one sensor can include a gas sensor. The gas sensor can be carried with the at least one shelf and enclosed by the chamber. The processing circuitry can perform signal-processing of at least one electrical signal received from the at least one sensor. For example, such signal-processing can help detect or measure a specified gas component or composition associated with the biological specimen.

The incubator can include at least one of a vessel transporter or a gas sensor transporter communicatively coupled with the processing circuitry to move at least one of the at least one gas sensor or an individual biological specimen vessel to be in communication with each other. For example, the vessel transporter can include a carousel for moving the individual vessels along a carousel route such that at least one vessel in a series of vessels in the carousel can be placed in communication with the at least one gas sensor. The incubator can also include or use a specimen agitator, coupled to the at least one shelf for moving the shelf such as to agitate at least one specimen vessel. The incubator can include a thermostat to regulate a temperature of the gas environment contained within the chamber.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts an example of a biological specimen incubator.

FIG. 1B depicts a cross-section of the biological specimen incubator of FIG. 1A across the plane 1B-1B.

FIG. 2A is a block diagram depicting an example of an incubation system.

FIG. 2B is a block diagram depicting an example of an incubation system.

FIG. 3 is a flowchart that describes a method for incubating a biological specimen using an example of an incubation system.

FIG. 4 is a block diagram illustrating components of a device or machine to read instructions from a machine-storage medium (e.g., a non-transitory machine-storage medium, a machine-storage medium, a computer-storage medium, or any suitable combination thereof) and perform any one or more of the methodologies discussed herein, in whole or in part.

### DETAILED DESCRIPTION

Certain biological specimen incubation techniques can be used such as to determine a presence or other characteristic of a target foreign body within a biological specimen. For example, a plurality of different biological specimens can be placed into an incubator, each for an incubation period. During the incubation period, a target foreign body within an individual biological specimen can become detectable. Following incubation, the biological specimens can be removed and assayed using one or more specific detection techniques.

One approach to biological specimen incubation involves visual inspection of biological specimens for signs of an infection, such as observing for signs of a change in color, clarity, size, or other characteristics of the specimen. Here, “visual” inspection can refer to inspection of characteristics visible to a himan observer at wavelengths visible to the human eye. Such visual inspection can present certain challenges, such as the difficulty in inspecting the entire specimen surface, such as to determine a presence of an infectious agent or other foreign body, and possible operator-introduced errors. Also, such an approach can involve manual intervention, such as manual agitation of a sample, during incubation or in between incubation sessions. Such manual intervention can present challenges, such as difficulty in obtaining repeatable results and possible operator-introduced errors.

In another approach to biological specimen incubation, a sensor can be used to obtain some of the information necessary for determining the presence or other characteristic of the target foreign body within the biological specimen. However, certain approaches involving a sensor can increase the time required to perform the incubation as compared with some manual techniques. Further, the use of such sensors can require a relatively high level of expertise to configure and operate the system and can be relatively expensive and difficult to scale up.

This document describes, among other things, an automated biological specimen incubation system that addresses at least some of the challenges of other approaches discussed above. A blood incubation system can include an incubator to automatically agitate a biological specimen, such as at a specified and controllable rate. The incubator can also control, maintain, or modulate the temperature of the biological specimen during incubation. The incubator can include or use at least one active shelf such as to provide an electrical or optical signal, e.g., from an embedded sensor within a biological specimen vessel, to analysis circuitry. The incubator can also include or use a mechanism for controlling the placement or positioning of culture samples such as with respect to at least one sensor.

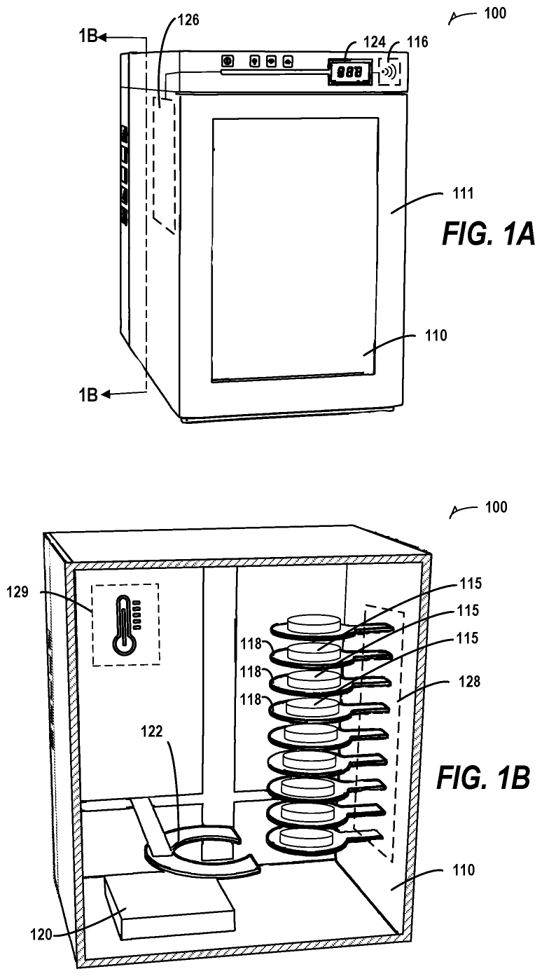

FIG. 1A depicts an example of a biological specimen incubator 100. FIG. 1B depicts a cross-section of the biological specimen incubator 100 of FIG. 1A across the plane 1B-1B. The biological specimen incubator 100 can include an incubator chamber 110 and at least one imaging or other sensor 120. The incubator chamber 110 can be accessed, e.g., via a door 111, such as to introduce biological specimen vessels 115 into the chamber 110. For example, the biological specimen vessels 115 can include any of a Petri dish, a multiwell plate, a microtiter plate, a chip, or a slide. The biological specimen vessel may include a tube, such as a tube formed from a heat sealable plastic, for example. Additionally, the biological specimen vessel 115 may include a well, groove, well-plate, strip, pad, and other suitable container that holds at least one specimen of a biological material. Once the vessels 115 are introduced, the incubator chamber 110 can be fluidly sealed off from an ambient environment, thereby enclosing the vessels 115. The chamber 110 can include or use one or more shelves 118. In an example, an individual shelf 118 can one or multiple receptacles for respectively receiving biological a respective specimen vessel.

At least one of: a) an individual biological specimen vessel 115 (e.g., as depicted in FIG. 1B) or b) the imaging or other sensor 120 can be movable with respect to the other, such as via a manipulator or transporter 122. The manipulator 122 can place at least one of: a) an individual biological specimen vessel 115 or b) the imaging or other sensor 120 in communication with the other, such as for imaging of the respective specimen using the imaging or other sensor 120. The manipulator 122 can include a robotic mechanism such as a gantry, an articulating arm, or an articulated platform. The manipulator 122 can include at least one placement sensor for providing feedback in placement using the manipulator 122. For example, the at least one placement sensor can include an optical sensor, and the placement feedback can include one or more LEDs or other light sources emitting energy and one or more photodetectors that detect the emitted energy. The at least one placement sensor can also include one or more capacitive sensors, conductive sensors, IR sensors, or RF sensors, cameras, or combinations thereof.

The at least one imaging or other sensor 120 can generate and transmit a signal that can include information representing a measurement of a concentration or other characteristic of a specified gas component or composition associated with a target biological specimen in an individual one of the specimen vessels 115. The signal produced by the imaging or other sensor 120 can be signal-processed or analyzed, e.g., based on an electro-chemical detection characteristic or an electro-optical detection characteristic measured by the imaging or other sensor 120 within the chamber 110. For example, the imaging or other sensor 120 can include an imaging array of photosensitive elements configured to detect electromagnetic energy within the specified wavelength band, e.g., visible light or the near-infrared spectrum. Also, for example, the imaging or other sensor 120 can include a solid-state imaging array of detector pixels that can be configured to detect specific fluorescence emissions or other optical signatures from the target biological specimen. Other examples of imaging sensors 120 can include electro-chemical sensing systems configured to detect the presence of a gas component or gas components in the specimen fluid. These can be used to sense the presence or other characteristic of a target gas component of interest or a gas component that affects the optical or electro-chemical signatures from the target biological specimen. In an example, the at least one imaging or other sensor 120 can include a plurality of gas sensors each respectively positionable or locatable in communication with a respective vessel 115 or receptacle of the plurality of receptacles.

A reading of the measurement from the imaging or other sensor 120 can be communicated to and provided at a location outside of the incubator 100, such as at a user interface (UI) 124. For example, the UI 124 can include a display to display the result of the measurement, e.g., the type or concentration of a target gas component detected by the imaging or other sensor 120, or an interpretation of the measurement, e.g., the presence or concentration of a target substance in the vessel 115 represented by the sensor detection. Also, the UI 124 can include other output means, e.g., a speaker, a speaker unit, a vibrator, a buzzer, or other similar output means. The incubator 100 can include transceiver circuitry 116 for transmitting the electrical response signal or the determined presence or other characteristic to a location outside the chamber 110. For example, the transceiver circuitry 116 can include an external receiver and a wired or wireless communication link to a device that is external to the chamber 110, such as to a local or remote computer system that is used to perform computational analysis. Also, for example, the transceiver circuitry 116 can include a communication link to an external device that can be used to perform additional processing to that performed by onboard processing circuitry, such as control of the temperature or pressure of the gas environment contained within the chamber 110.

The incubator 100 can include a thermostat 129 to regulate a temperature of the gas environment contained within the chamber 110. The thermostat 129 can include, e.g., an analog, thermistor, or thermocouple type temperature sensor or electronic temperature sensor configured to determine a temperature. The temperature sensor can be included in communication with a heating and cooling unit to control the temperature of the gas environment within the chamber 110. In an example, the heating and cooling unit can include a plurality of heating elements configured to heat a gas environment to a desired temperature. In an example, the heating and cooling unit can include a cooling element, e.g., a Peltier cooling element, to cool the gas environment to a desired temperature. Thus, in an example, the chamber 110 can include a thermostat configured to maintain a desired temperature by activating one or more heating elements when a temperature of the chamber 110 is below a specified level and activating one or more cooling elements when a temperature of the chamber 110 is above the specified level.

The incubator 100 can include an agitator 128, e.g., coupled to the at least one shelf 118 for moving the shelf 118 and agitating the biological specimen vessels carried thereon. The agitator 128 can be used to move liquid in the vessel, thereby causing movement in the biological specimen. The agitator 128 can include, e.g., a vibrating agitator, a rocking agitator, a reciprocal linear agitator, a reciprocating linear agitator, and a reciprocating rotary agitator, any of which can be operated by a motor or by manual operation. For example, the reciprocating linear agitator can include a motor for rotating the reciprocating linear agitator, such as a motor with an elongated rod and an eccentric weight at its end. In another example, a motor can be used to reciprocate the linear agitator along its length. Alternatively or additionally, the manipulator 122 can be used for agitating, such as to agitate an individual specimen vessel 115.

Processing circuitry 126 can be included or used, such as onboard the incubator 100, to regulate the temperature or pressure environment of the chamber 110. The processing circuitry 126 can also position the at least one imaging or other sensor 120 relative to the target biological specimen in the specimen vessel 115, or to regulate the position of the at least one manipulator 122. The processing circuitry 126 can include a processor circuit and a memory circuit that can store a program or a series of programs for instructing the processor to carry out the processing steps, such as to maintain a selected temperature and pressure condition within the chamber 110, to detect the presence or other characteristic of the target gas component in the biological specimen, and to perform or coordinate other steps, e.g., such as for transmitting the resulting measurement to the UI 124, controlling agitation via the agitator 128, regulating temperature via the thermostat 129, operating the manipulator 122, or communicating via the transceiver circuitry 116. The processing circuitry 126 can be used to run one or more programs written in a computer programming language, such as FORTRAN, BASIC, C, C++, or other suitable language. Furthermore, the processing circuitry 126 can include a plurality of processors and memory circuits for individually executing and storing the programs. The processing circuitry 126 can include an external interface that permits communication with devices, computers, and other programs that are external to the biological specimen incubator 100. For example, the external interface can include an RS-232 or RS-485 serial port, an ethernet interface, a modem, or a communication link to a network, such as a LAN or the Internet, to permit communication with a remote device or an Internet database. For example, the processing circuitry 126 can include a communications component, such as a serial-to-parallel converter or other interface component to facilitate such an external interface.

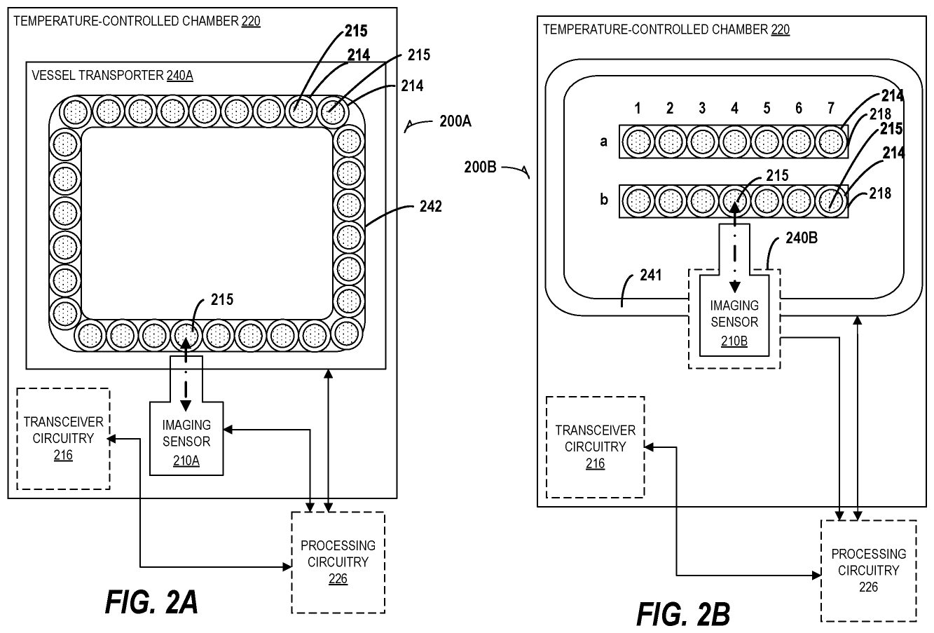

FIG. 2A and FIG. 2B are block diagrams each depicting an example of an incubation system. The incubation systems 200A and 200B can be similar to the incubator 100 described above. Thus, components, operations, features, and other portions of the incubator 100 can be incorporated into the incubation systems 200A and 200B.

FIG. 2A depicts an incubation system 200A that can include a temperature-controlled chamber 220, a vessel transporter 240A, an imaging sensor 210A, transceiver circuitry 216, and processing circuitry 226. In an example, the vessel transporter 240A can include a carousel 242 for moving a plurality of receptacles 214, each including a respective specimen vessel 215, along a carousel route. Here, the carousel 242 can position the plurality of receptacles 214 such that at least one vessel 215 in a series of vessels in the carousel 242 can be placed in communication with the at least one imaging sensor 210A. For example, the at least one imaging sensor 210A can remain stationary while specimens can be continuously positioned and presented to the sensor 210A, and imaging data can be received with respect to an individual specimen at a regular or semi-regular interval. The carousel route can extend such that, e.g., the carousel 242 can move in a substantially continuous path. Also, the carousel route can extend substantially in one direction, or substantially along an X-Y plane. For example, the carousel 242 can remain stationary while the receptacles (e.g., specimen vessels) along the carousel route are incubated. After incubation is complete or during a break in incubation, the carousel 242 can be moved to present the series of vessels back in communication with the at least one imaging sensor 210, thereby providing the received imaging data to the processing circuitry 226. Thereafter, the processing circuitry 226 can analyze the images captured from the at least one imaging sensor 210.

FIG. 2B depicts an incubation system 200B that can include a temperature-controlled chamber 220, a sensor transporter 240B, an imaging sensor 210B, at least one shelf 218, transceiver circuitry 216, and processing circuitry 226. In an example, the sensor transporter 240B can be attached to the imaging sensor 210B to move the sensor 210B with respect to the plurality of receptacles 214 along a sensor route 241. Here, a specified receptacle of the plurality of receptacles 214 can be positioned proximate the imaging sensor 210B to allow imaging data to be received from the specimen in the specified receptacle. For example, the sensor transporter 240B can manipulate the imaging sensor 210B such as to sequentially place the sensor 210B in communication with a plurality of receptacles 214, such as in columns 1-7, such as one at a time. The sensor transporter 240B can manipulate the imaging sensor 210B such as to sequentially place the sensor 210B in communication with a plurality of receptacles located on a plurality of different shelves 218, such as shelves depicted by rows a and b. For example, the processing circuitry 226 can select an individual receptacle of rows a and b and columns 1-7, such as receptacle “b, 4” as depicted in FIG. 2B. After a specified receptacle has been placed in communication with the imaging sensor 210B, the processing circuitry 226 can perform an analysis with respect to the imaging data received from the imaging sensor 210B.

An individual receptacle 214 can include at least one power connection to interface with a respective vessel inserted therein. For example, the receptacle 214 can include a power connection to provide power and/or instructions to control the incubation procedure within the respective receptacle. Also, an individual receptacle 214 can include at least one sensor connection such as for interfacing with an auxiliary sensor included within an individual respective specimen vessel 215.

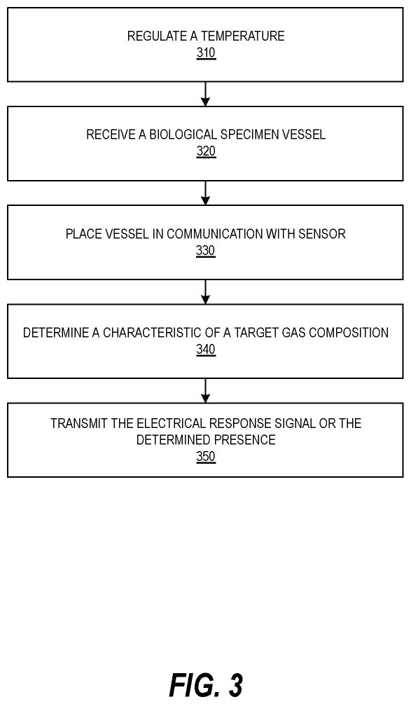

FIG. 3 is a flowchart that describes a method for incubating a biological specimen using an example of an incubation system. At 310, the method can include regulating a temperature within an incubation chamber. Also, the biological specimen can be agitated such as to promote culture growth within the specimen. At 320, a biological specimen vessel can be received within an individual receptacle of a plurality of receptacles included in a shelf of the chamber.

At 330, a biological specimen vessel can be placed in communication with an imaging sensor for generating an electrical response signal indicating an electro-chemical characteristic associated with the specimen. This can include moving at least one of the biological specimen vessels or the gas sensor with respect to the other one of the individual vessels or the gas sensor. Placing can include moving a plurality of biological specimen vessels along a carousel route such that at least one vessel in a series of vessels along the carousel route can be placed in communication with the gas sensor. Placing can also include moving the gas sensor towards an individual receptacle of the plurality of receptacles.

At 340, the method can include sensing and determining, using at least one electrical response signal, a presence or other characteristic of at least one target gas composition associated with the biological specimen. For example, the at least one electrical sensor signal can be signal processed for detecting and measuring a specified gas component or composition. At 350, the electrical response signal or extracted information about the determined presence or other characteristic can be transmitted to a a location outside the chamber.

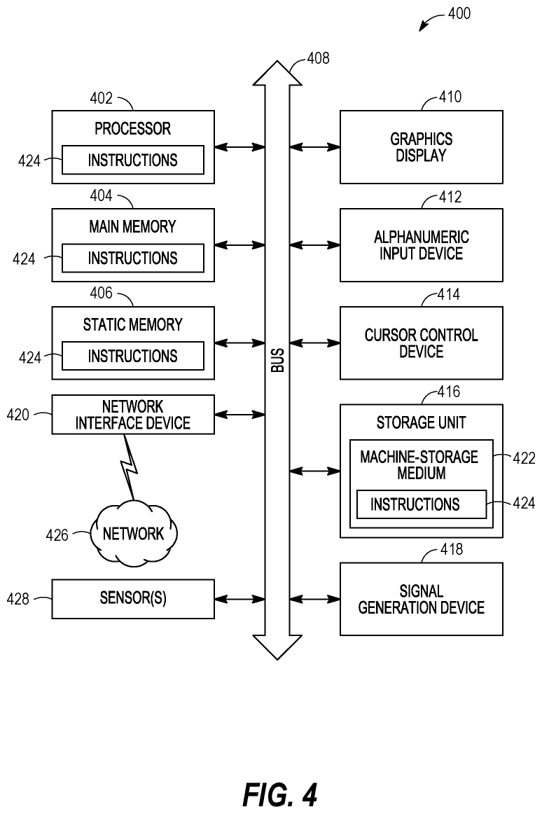

FIG. 4 is a block diagram illustrating components of a machine 400, according to some example embodiments, able to read instructions 424 from a machine-storage medium 422 (e.g., a non-transitory machine-storage medium, a machine-storage medium, a computer-storage medium, or any suitable combination thereof) and perform any one or more of the methodologies discussed herein, in whole or in part. Specifically, FIG. 4 shows the machine 400 in the example form of a computer system (e.g., a computer) within which the instructions 424 (e.g., software, a program, an application, an applet, an app, or other executable code) for causing the machine 400 to perform any one or more of the methodologies discussed herein can be executed, in whole or in part. For example, the instructions 424 can be processor executable instructions that, when executed by a processor of the machine 400, cause the machine 400 to perform the operations outlined above.

In various embodiments, the machine 400 operates as a standalone device or can be communicatively coupled (e.g., networked) to other machines. In a networked deployment, the machine 400 can operate in the capacity of a server machine or a client machine in a server-client network environment, or as a peer machine in a distributed (e.g., peer-to-peer) network environment. The machine 400 can be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any machine capable of executing the instructions 424, sequentially or otherwise, that specify actions to be taken by that machine. Further, while only a single machine is illustrated, the term “machine” shall also be taken to include any collection of machines that individually or jointly execute the instructions 424 to perform all or part of any one or more of the methodologies discussed herein.

The machine 400 includes a processor 402 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 404, and a static memory 406, which are configured to communicate with each other via a bus 408. The processor 402 can contain microcircuits that are configurable, temporarily or permanently, by some or all of the instructions 424 such that the processor 402 is configurable to perform any one or more of the methodologies described herein, in whole or in part. For example, a set of one or more microcircuits of the processor 402 can be configurable to execute one or more modules (e.g., software modules) described herein.

The machine 400 can further include a graphics display 410 (e.g., a plasma display panel (PDP), a light emitting diode (LED) display, a liquid crystal display (LCD), a projector, a cathode ray tube (CRT), or any other display capable of displaying graphics or video). The machine 400 can also include an alphanumeric input device 412 (e.g., a keyboard or keypad), a cursor control device 414 (e.g., a mouse, a touchpad, a trackball, a joystick, a motion sensor, an eye tracking device, or other pointing instrument), a storage unit 416, an audio generation device 418 (e.g., a sound card, an amplifier, a speaker, a headphone jack, any suitable combination thereof, or any other suitable signal generation device), and a network interface device 420.

The storage unit 416 includes the machine-storage medium 422 (e.g., a tangible and non-transitory machine-storage medium) on which are stored the instructions 424, embodying any one or more of the methodologies or functions described herein. The instructions 424 can also reside, completely or at least partially, within the main memory 404, within the processor 402 (e.g., within the processor's cache memory), or both, before or during execution thereof by the machine 400. Accordingly, the main memory 404 and the processor 402 can be considered machine-storage media (e.g., tangible and non-transitory machine-storage media). The instructions 424 can be transmitted or received over the network 426 via the network interface device 420. For example, the network interface device 420 can communicate the instructions 424 using any one or more transfer protocols (e.g., Hypertext Transfer Protocol (HTTP)).

In some example embodiments, the machine 400 can be a portable computing device, such as a smart phone or tablet computer, and have one or more additional input components (e.g., sensors 428 or gauges). Examples of the additional input components include an image input component (e.g., one or more cameras), an audio input component (e.g., a microphone), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components can be accessible and available for use by any of the modules described herein.

### Executable Instructions and Machine-Storage Medium

The various memories (i.e., 404, 406, and/or memory of the processor(s) 402) and/or storage unit 416 can store one or more sets of instructions and data structures (e.g., software) 424 embodying or utilized by any one or more of the methodologies or functions described herein. These instructions, when executed by processor(s) 402 cause various operations to implement the disclosed embodiments.

As used herein, the terms “machine-storage medium,” “device-storage medium,” “computer-storage medium” (referred to collectively as “machine-storage medium 422”) mean the same thing and can be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 422 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage medium or media, computer-storage medium or media, and device-storage medium or media 422 specifically exclude carrier waves, modulated data signals, and other such media, at least some of which are covered under the term “signal medium” discussed below. In this context, the machine-storage medium is non-transitory.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer Readable Medium

The terms “machine-readable medium,” “computer-readable medium” and “device-readable medium” mean the same thing and can be used interchangeably in this disclosure. The terms are defined to include both machine-storage media and signal media. Thus, the terms include both storage devices/media and carrier waves/modulated data signals.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An incubation system for gas detection of at least one incubated biological specimen, the system comprising:
at least one gas sensor, arranged to be placed in communication with at least one biological specimen vessel for generating at least one of an electrical response signal or an optical response signal indicating an attribute associated with a particular biological specimen;
a temperature-controlled chamber, including within the chamber at least one shelf defining a plurality of receptacles, an individual receptacle sized and shaped for receiving an individual vessel of the at least one biological specimen vessel;
processing circuitry configured to:
place the at least one gas sensor and the individual vessel in communication with each other, the individual vessel configured for carrying the particular biological specimen; and
determine, based on the attribute indicated by the at least one of an electrical response signal or an optical response signal, at least one of a presence of, or a chemical characteristic of, at least one target gas composition associated with the particular biological specimen; and
transceiver circuitry for transmitting the at least one of an electrical response signal or an optical response signal or the determined presence or chemical characteristic to a location outside the chamber.

2. The system of claim 1, wherein the at least one gas sensor is carried with the at least one shelf and enclosed by the chamber.

3. The system of claim 1, wherein the at least one gas sensor includes a plurality of gas sensors each respectively locatable in communication with a respective receptacle of the plurality of receptacles.

4. The system of claim 3, wherein an individual receptacle of the plurality of receptacles includes electrical circuitry in wired or wireless communication with at least one of:
the respective gas sensor in communication with the individual receptacle; or
the at least one biological specimen vessel received within the individual receptacle.

5. The system of claim 1, comprising at least one of a vessel transporter or a gas sensor transporter communicatively coupled with the processing circuitry to move at least one of the at least one gas sensor or an individual biological specimen vessel to be in communication with each other.

6. The system of claim 5, comprising a vessel transporter including a carousel for moving the individual vessels along a carousel route such that at least one vessel in a series of vessels in the carousel is placed in communication with the at least one gas sensor.

7. The system of claim 5, comprising a gas sensor transporter configured to move the at least one gas sensor towards an individual receptacle of the plurality of receptacles.

8. The system of claim 5, wherein the processing circuitry is configured to operate at least one of the vessel transporter or the gas sensor transporter to:
identify an individual biological specimen vessel including a particular biological specimen;
place the identified individual biological specimen vessel in communication with the at least one gas sensor; and
obtain a measurement of a concentration of a specified gas component or composition associated with a biological specimen carried within the individual biological specimen vessel.

9. The system of claim 1, comprising an agitator, coupled to the shelf for moving the shelf for agitating the biological specimen vessels.

10. The system of claim 1, comprising a thermostat configured to regulate a temperature of fluid contained within the chamber.

11. The system of claim 1, wherein the transceiver circuitry includes a wireless transmitter for providing a wireless communication between the gas sensor and a remote receiver.

12. The system of claim 1, wherein the transceiver circuitry includes wired connection circuitry for providing a wired communication between the gas sensor and a remote receiver.

13. A method for incubating a biological specimen, the method comprising:
regulating a temperature within an incubation chamber, the chamber including within the chamber at least one shelf defining a plurality of receptacles, an individual receptacle sized and shaped for receiving an individual vessel of the at least one biological specimen vessel
receiving a biological specimen vessel within an individual receptacle of a plurality a receptacles included in a shelf of the chamber;
placing the biological specimen vessel in communication with a gas sensor for generating an electrical response signal indicating an electro-chemical characteristic associated with the specimen;
determining, using the at least one electrical response signal, a presence or other characteristic of at least one target gas composition associated with the biological specimen; and
transmitting the electrical response signal or the determined presence or other characteristic to a location outside the chamber.

14. The method of claim 13, comprising signal-processing the at least one electrical signal for detecting and measuring a specified gas component or composition associated with the biological specimen.

15. The method of claim 13, comprising moving the shelf for agitating the biological specimen vessel.

16. The method of claim 13, comprising moving at least one of the biological specimen vessel or the gas sensor with respect to the other one of the individual vessel or the gas sensor.

17. The method of claim 16, wherein moving includes moving a plurality of biological specimen vessels along a carousel route such that at least one vessel in a series of vessels along the carousel route is placed in communication with the gas sensor.

18. The method of claim 16, wherein moving includes moving the gas sensor towards an individual receptacle of the plurality of receptacles.

19. A biological specimen incubator comprising:
an incubator chamber, including within the chamber a plurality of shelves, an individual shelf defining a respective plurality of receptacles for receiving biological specimen vessels; and
a gas sensor, locatable in communication with an individual one of the biological specimen vessels for obtaining a measurement of a concentration of a specified gas component or composition associated with biological specimen in the individual one of the specimen vessels based on an electro-chemical detection characteristic or an electro-optical detection characteristic measured by the gas sensor within the chamber, and providing a reading of the measurement at a location outside of the incubator.

20. The incubator of claim 19, further comprising moving the gas sensor from a first biological specimen vessel within a receptacle of a first shelf towards a second biological specimen vessel within a receptacle of a second shelf.
